(19) United States
(12) Patent Application Publication
(10) Pub. No.: US 2011/0004163 A1
(43) Pub. Date: Jan. 6, 2011

(54) METHOD AND APPARATUS FOR INTRAOSSEOUS INFUSION

(76) Inventor: Hrushikesh U. Vaidya, Hays, KS (US)

Correspondence Address: HURLEY STANNERS, LLC, 1011 LAKE STREET, OAK PARK, IL, 60301-1137, US

(21) Appl. No.: 12/496,209

(22) Filed: Jul. 1, 2009

(51) Int. Cl. A61B 17/34; A61M 25/02

(52) U.S. Cl. 604/164.04

(57) ABSTRACT

A method, apparatus and kit for an intraosseous infusion of a body is disclosed. In one embodiment, the apparatus includes a trocar, a triple lumen, and a handle. In another embodiment, a method is disclosed, the method including the steps of inserting the trocar and lumen, removing the trocar, inserting a triple lumen, and infusing the body with fluids.

### FIELD OF THE INVENTION

The present invention relates generally to a method and apparatus for infusing fluids into a body, and in particular, the present invention relates to intraosseous infusion of fluids.

### BACKGROUND OF THE INVENTION

In the medical field, it is often necessary to introduce fluids into a body, such as through an intra venous (IV) line or through some other type of port that can direct necessary fluids to a portion of a person's body. If configured properly, such a line may also be used to remove or suction fluids from the body as needed, as well.

### SUMMARY OF THE INVENTION

The present invention relates to one or more of the following features, elements or combinations thereof. An apparatus having a handle and an outer cannula is disclosed. A trocar is fitted inside the outer cannula and can be used to puncture tissue in a body, for example skeletal tissue. The trocar can also be replaced with a multi-passage lumen for infusion or removal of fluids from the body. A method of using the apparatus is also disclosed.

### BRIEF DESCRIPTION OF THE DRAWINGS

FIG. 1 is a sectional view of one embodiment of the disclosed intraosseous infusion device;

FIG. 2 is a top view of a flap C of FIG. 1;

FIG. 3 is a perspective view of collar D of FIG. 1;

FIG. 4 is a cross-sectional view taken through the line 4-4 of FIG. 3;

FIG. 5 is a cross-sectional view taken through the line 5-5 of FIG. 3;

FIG. 6 is a view of the inner trocar used with the infusion device;

FIG. 7 is a perspective view of a lumen that is configured to be used with the intraosseous infusion device;

FIG. 8 is a cross-sectional view of the lumen of FIG. 7, taken through the line 8-8;

FIG. 9 is a perspective view of the lumen head and connection portion;

FIG. 10 is a perspective view of a luer connector;

FIG. 11 is a perspective view of the intraosseous infusion device having a trocar positioned therein; and

FIG. 12 is a perspective view of the intraosseous infusion device having a lumen positioned therein.

### DETAILED DESCRIPTION OF EMBODIMENTS OF THE INVENTION

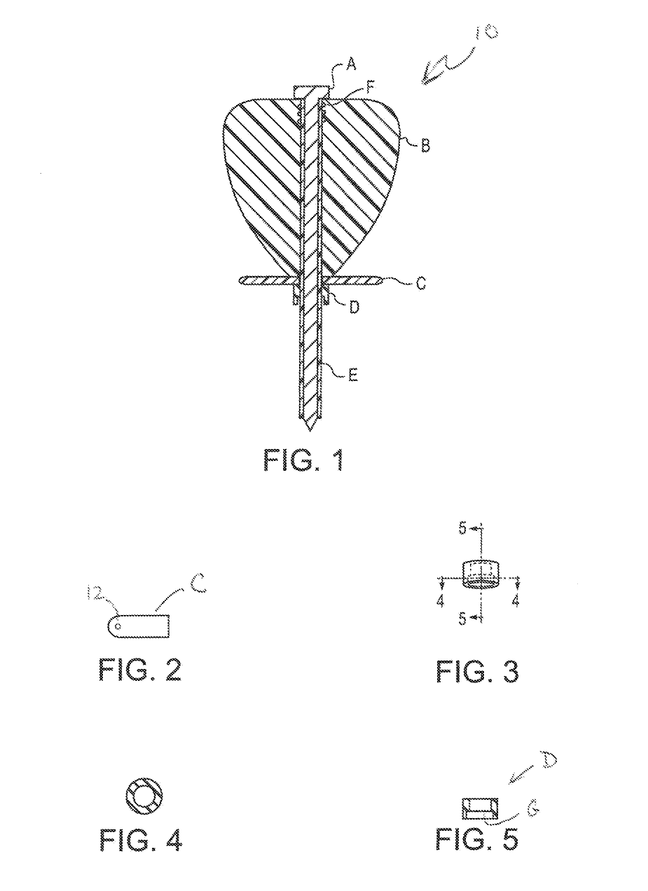

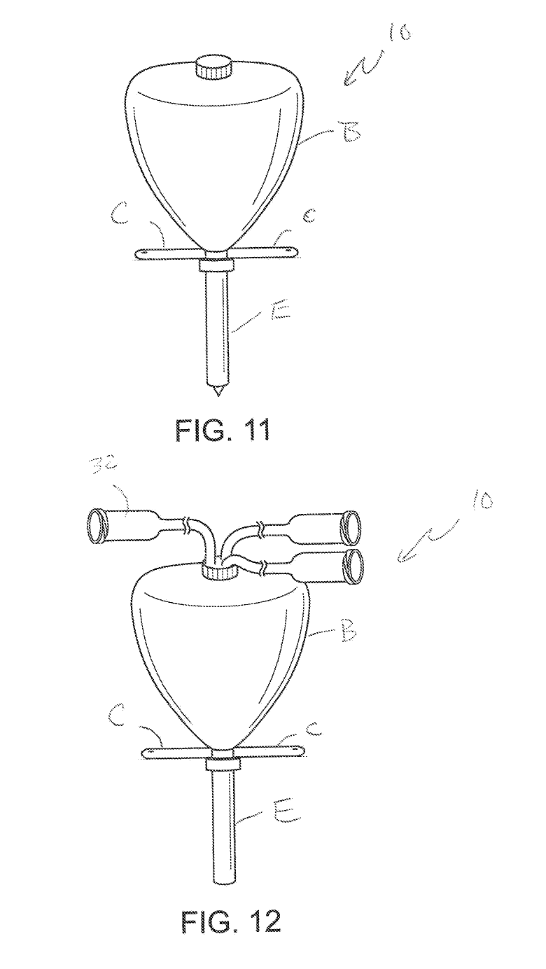

A kit or apparatus 10 for use in transmitting fluids to or from a body, such as a person, is shown in cutaway view in FIG. 1 and in perspective view in FIGS. 11 and 12. Illustratively, the apparatus 10 can be used for the intraosseous infusion of fluids. In the illustrated embodiment, the apparatus 10 generally comprises a handle B, a trocar A, and an outer cannula E that encompasses the outer portion of the trocar. Handle B may be formed of rubber, plastic, metal, or any other material that is suitable to provide a grip and may be contoured or grooved in order to provide a surface that does not slip when gripped. Outer cannula E may also be formed of a hard material, such as plastic or metal. Cannula E may be configured to be a particular length, so as to be long enough to penetrate a selected bone and remain fixed in the bone, yet not so long that it penetrates through the entire bone. It is contemplated that cannula E can be manufactured of varying lengths and thicknesses per the use demanded.

Typically, trocar A is formed of metal but may also be formed of any hard material capable of penetrating bone. Illustratively, trocar A has a bone-cutting edge.

In the embodiment shown in FIGS. 1, 11, and 12, flaps C are also shown. Such flaps are one embodiment of a means for securing apparatus 10 to a body. In the illustrated example, flaps C are configured to have an aperture 12, shown in FIG. 2, such that the flaps C can be sutured to tissue, thereby helping to hold the apparatus 10 in place once it is positioned. Such flaps C may be made of a flexible or solid material, such as plastic. However, it is contemplated that other materials would also suffice. Illustratively, the flaps C are connected to handle B such that the flaps can hold outer cannula E in its position.

FIGS. 3, 4, and 5 illustrate a collar D that is mounted on outer cannula E. Illustratively, collar D and outer cannula E are connected. It is contemplated that collar D and outer cannula E can be formed separately and later joined. Collar D is formed such that it has a recess G formed on a surface that would face bone tissue, so as to provide suction against the bone and assist in holding apparatus 10 in position after it is placed. Moreover, collar D functions as a stop when outer cannula E is inserted into a body. Collar D may be formed of semi-soft rubber or silicone. It is of predetermined length and functions to apply a negative pressure against bone. It also functions to prevent fluid from coming back through the bone. Collar D also functions to provide a specific length of cannula E that is exposed for penetration into the bone. For example, with a body that has significant subcutaneous tissue, such as in heavier individuals, an incision can be made into the tissue of the body such that collar D rests up against the bone and is lodged within the subcutaneous tissue, thereby holding collar D and cannula E in place.

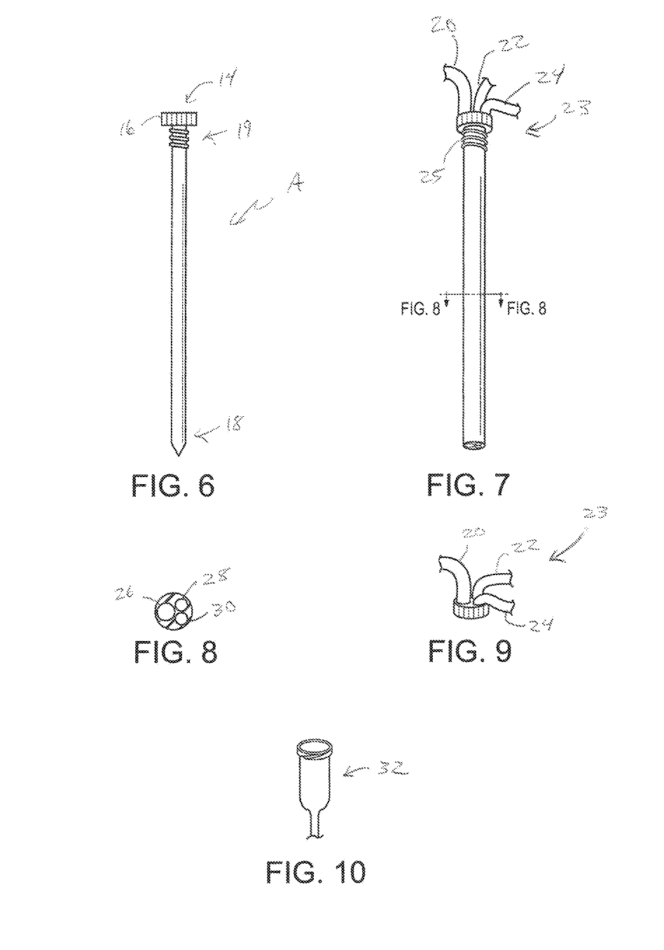

FIG. 6 shows trocar A after it has been removed from its position inside outer cannula E. Illustratively, trocar A has a head 14 having grooves 16 formed thereon, for facilitating the insertion and removal of trocar A from the body. Trocar A may have a distal end 18 that is configured to puncture bone. For example, trocar A may be a three-sided so that the trocar may be forced axially into a bone or urged with a twisting motion so as to direct trocar A into the bone. Trocar A also has a male luer portion 19 for connecting with threaded portion F of outer cannula E.

FIG. 7 shows a perspective view of a lumen 21 that can be inserted in the place of trocar A in outer cannula E. In this illustration, lumen 21 has a head end 23 having a luer portion 25. The head end 23 is further configured to connect to three fluid conduits 20, 22, and 24. A cross-sectional view of lumen 21 is shown in FIG. 8, which also illustrates three passages 26, 28, and 30, which are each configured to transport fluids either to or away from a body. Illustratively, conduit 20 (and likewise passage 26) is larger in size than conduits 22 and 24 (and passages 28 and 30). However, varying sizes of conduits and passages are within the scope of the disclosure. FIG. 9 shows another perspective view of the head end 23 of lumen 21.

FIG. 10 shows a female luer lock 32 that might be attached at a distal end of any of the conduits 20, 22, 24. Such a luer lock permits a syringe to be inserted, and also permits engagement of a fluid line with each of the three conduits 20, 22, 24. A perspective view of the apparatus 10 with luer locks 32 attached to fluid conduits 20, 22, 24 is shown in FIG. 12.

Apparatus 10 is utilized in substantially the following fashion. A patient or body is determined to be in need of an intraosseous infusion. An incision can be made in the body so as to open the body and expose the bone through which the apparatus 10 will be placed. In the case of an emergency, it is possible to directly insert apparatus 10 without first making an incision.

Apparatus 10 is held by a physician by handle B. The physician places distal end 18 of trocar A proximal to a selected bone (i.e. the tibia) and inserts the distal end 18 in a manner consistent with the use of trocars. For example, in some trocar embodiments, a physician may press against handle B of apparatus 10 so as to cause trocar A to puncture the bone. In other embodiments, a physician may use handle B to twist trocar A into the bone.

Once the distal ends of trocar A and outer cannula E are inserted in the body, trocar A can be removed from within outer cannula E. For example, trocar A can be unscrewed or removed with assistance of head 14 of trocar A. In such a scenario, head 14 may have grooves 16, shown in FIG. 6.

Once trocar A is removed, a triple lumen 21, such as that shown in FIG. 7 (having conduits 20, 22, and 24) can be inserted through outer cannula E. In the alternative, multiple conduits or lumens may be inserted individually. The disclosed triple lumen 21 can be used to either infuse fluids into the body or suction fluids from the body. As discussed above, conduits 20, 22, 24 could be of varying sizes depending on the needs and use of the triple lumen. FIG. 12 illustrates the apparatus 10 after the triple lumen has been inserted.

When there is a lack of pulse due to cardiac rest, it is still important to provide fluids, and life-saving drugs in some fashion. A device with multiple lumens, such as that disclosed, allows for a number of fluids to be provided at the same time without interruption. This also allows for a higher flow rate of fluids.

While the various steps set forth above have been described in a particular order, it should be understood that the invention is not limited to this particular order or to all of these steps being performed. Rather, other orders are contemplated and may be utilized.

While the disclosure is susceptible to various modifications and altercations and alternative forms, specific exemplary embodiments thereof have been shown by way of example in the drawings and therein been described in detail. It should be understood, however, that there is not intent to limit the disclosure to the particular forms disclosed, but on the contrary, the intention is to cover all modifications, equivalents, and alternatives falling within the spirit and scope of the disclosure as defined by the appended claims.

WHAT IS CLAIMED IS:

1. An apparatus for the intraosseus infusion of fluids into a body, the apparatus comprising:
a trocar;
an outer cannula encompassing at least a portion of the trocar, the outer cannula having a pre-selected length;
a handle connected to the outer cannula; and
a means for securing the apparatus to the body, the securing means being connected to the handle.

2. The apparatus of claim 1, wherein the trocar is movable relative to the outer cannula.

3. The apparatus of claim 1, wherein the trocar is configured to be removed from contact with the outer cannula.

4. The apparatus of claim 1, further comprising a collar mounted on the outer cannula.

5. The apparatus of claim 4, wherein the collar is configured to be positioned adjacent a bone of a body.

6. The apparatus of claim 1, wherein the securing means comprises at least one flap.

7. The apparatus of claim 6, wherein the flap has an aperture formed therein for receiving a suture.

8. The apparatus of claim 6, wherein the flap is configured to be sutured to the body.

9. The apparatus of claim 1, further comprising a lumen configured to be inserted in the outer cannula.

10. The apparatus of claim 9, wherein the lumen and trocar are exchangeable.

11. The apparatus of claim 9, wherein the lumen comprises at least three fluid conduits.

12. The apparatus of claim 11, wherein each of the at least three fluid conduits has a diameter, and one of conduit diameters is larger than the other conduit diameters.

13. The apparatus of claim 9, wherein the lumen has a luer portion.

14. An kit for use in the intraosseus infusion of fluids into a body, the kit comprising:
a trocar;
an outer cannula encompassing at least a portion of the trocar;
a handle connected to the outer cannula; and
a lumen capable of being inserted in the outer cannula.

15. The kit of claim 14, wherein the trocar is movable relative to the outer cannula.

16. The kit of claim 14, wherein the trocar is configured to be removed from contact with the outer cannula and the lumen is configured to be positioned in place of the trocar.

17. The kit of claim 14, further comprising a collar mounted on the outer cannula.

18. The kit of claim 17, wherein the collar is configured to be positioned adjacent a bone of a body.

19. The kit of claim 14, wherein the securing means comprises at least one flap.

20. The kit of claim 19, wherein the flap has an aperture formed therein for receiving a suture.

21. The kit of claim 19, wherein the flap is configured to be sutured to the body.

22. The kit of claim 14, wherein the lumen and trocar are exchangeable.

23. The kit of claim 14, wherein the lumen comprises at least three fluid conduits.

24. The kit of claim 23, wherein each of the at least three fluid conduits has a diameter, and one of conduit diameters is larger than the other conduit diameters.

25. The kit of claim 14, wherein the lumen has a luer portion.

26. The kit of claim 14, further comprising a means for securing the outer cannula to the body.
